(19) United States
(12) Patent Application Publication
(10) Pub. No.: US 2026/0055177 A1
(43) Pub. Date: Feb. 26, 2026

(54) SYNTHETIC EXTRACELLULAR INTERLEUKIN 23 BIOSENSORS

(71) Applicants: Northwestern University, Evanston, IL (US); The Regents of the University of Michigan, Ann Arbor, MI (US)

(72) Inventors: Joshua N. Leonard, Wilmette, IL (US); William Corcoran, Chicago, IL (US); Aaron Morris, Ann Arbor, MI (US)

(73) Assignees: Northwestern University, Evanston, IL (US); The Regents of the University of Michigan, Ann Arbor, MI (US)

(21) Appl. No.: 19/237,257

(22) Filed: Jun. 13, 2025

Related U.S. Application Data
(60) Provisional application No. 63/660,310, filed on Jun. 14, 2024.

(51) Int. Cl. C07K 16/24; C07K 14/705; C07K 14/71

(52) U.S. Cl. CPC C07K 16/244; C07K 14/70521; C07K 14/71; C07K 2317/569; C07K 2317/92; C07K 2319/03

(57) ABSTRACT

The present disclosure relates generally to the field of synthetic receptors and their uses. More specifically, the present disclosure relates novel synthetic receptors that target IL-23 and the use of such receptor in the treatment of conditions involving immune dysfunction, particularly autoimmunity.

### CROSS-REFERENCE TO RELATED APPLICATIONS

The present application claims priority to U.S. Provisional Application No. 63/660,310, filed Jun. 14, 2024, which is incorporated herein by reference in its entirety.

### STATEMENT OF GOVERNMENT SUPPORT

This invention was made with government support under Grant No. EB028840 from the NIH to A.H.M. The United States government has certain rights in the invention.

### SEQUENCE LISTING

The instant application contains a Sequence Listing which has been submitted electronically in XML format and is hereby incorporated by reference in its entirety. Said XML copy, created on Sep. 23, 2025, is named 121384-0275_SL.xml and is 12,676 bytes in size.

### TECHNICAL FIELD

The present disclosure relates generally to the field of synthetic receptors and their uses. More specifically, the present disclosure relates novel synthetic receptors that target IL-23 and the use of such receptor in the treatment of conditions involving immune dysfunction, particularly autoimmunity.

### BACKGROUND

The following description of the background of the present technology is provided simply as an aid in understanding the present technology and is not admitted to describe or constitute prior art to the present technology.

Early demonstrations of genetically engineering customized functions in mammalian cells indicate a vast potential to benefit applications including directed stem cell differentiation and cancer immunotherapy. In general, most applications require precise control of gene expression and the capability to sense and respond to external cues. Despite the growing availability of biological parts (such as libraries of promoters and regulatory proteins) that could be used to control cell states, assembling parts to compose customized receptors that function as intended remains a challenge.

Interleukin 23 (IL-23) is an inflammatory cytokine secreted by activated immune cells that plays a key role in the development of inflammation and the progression/onset of cancer. Thus, the IL-23 regulatory pathway represents a potential therapeutic target for the treatment of inflammatory diseases and cancer.

The present disclosure provides novel synthetic IL-23 biosensors that can be used in a variety of cell engineering platforms and other applications.

### SUMMARY

Described herein are synthetic receptor systems for engineering mammalian cell-based devices to sense soluble, physiological cues, including IL-23. More specifically, the present disclosure provides new receptor systems that combine a IL-23 extracellular ligand-binding domains with existing Modular Extracellular Sensor Architecture (MESA) to produce highly selective and useful cell biosensors and systems.

In one aspect, the present disclosure provides receptor comprising, consisting of, or consisting essentially of a protein dimer comprising, consisting of, or consisting essentially of a first protein and a second protein that each comprises, consists of, or consists essentially of: (a) an IL-23 extracellular ligand-binding domain, (b) a transmembrane domain, (c) an intracellular dimerizing domain. In some aspects, the intracellular dimerizing domain of the first protein comprises, consists of, or consists essentially of a first half of a split protease. In some other aspects, the intracellular dimerizing domain of the second protein comprises, consists of, or consists essentially of (i) a complementary second half of the split protease, (ii) a protease cleavage site (PCS), and (iii) a transcription factor linked thereto.

In some embodiments, the split protease components reconstitute upon dimerization of the first protein and the second protein, cleaving the PCS and releasing the transcription factor.

In some embodiments, the first protein and second protein each further comprise, consist of, or consist essentially of a juxtamembrane domain comprising, consisting of, or consisting essentially of 5-12 amino acids connected to a cytoplasmic end of the transmembrane domain.

In some embodiments, the first protein, the second protein, or both further comprise, consist of, or consist essentially of a signal peptide. In some aspects, the signal peptide is derived from a human CD8a receptor or a human IgG variable heavy chain.

In some embodiments, the extracellular domain of the first protein comprises, consist of, or consists essentially of a first nanobody comprising, consisting of, or consisting essentially of a binding domain that specifically binds to IL-23, and the extracellular domain of the second protein comprises, consists of, or consists essentially of a second nanobody comprising, consisting of, or consisting essentially of a binding domain that specifically binds to IL-23. In some aspects, the first nanobody binds to a first IL-23 epitope and the second nanobody binds to the first IL-23 or a second IL-23 epitope. In some aspects, the first nanobody comprises, consists of, or consists essentially of an amino acid sequence selected from SEQ ID NO: 10-12, and wherein the second nanobody comprises, consists of, or consists essentially of an amino acid sequence selected from SEQ ID NOs: 10-12. In some aspects, the second nanobody comprises, consists of, or consists essentially of a different amino acid sequence than the first nanobody.

In some embodiments, the extracellular domain, the transmembrane domain, and the juxtamembrane domain are all derived from the same human protein. In yet another aspect, the extracellular domain, the transmembrane domain, and the juxtamembrane domain are derived from at least two different human proteins.

In some embodiments, the transmembrane domain is derived from a murine or human CD28 receptor or a FGFR1 receptor.

In some embodiments, the juxtamembrane domain comprises a flexible repeated sequence of glycine and serine amino acids.

In some embodiments, the first protein comprises, consists of, or consists essentially of an N-terminal half of a split tobacco etch virus protease and the second protein comprises, consists of, or consists essentially of a complementary C-terminal half of a split tobacco etch virus protease, a protease cleavage site (PCS), and a transcription factor. In yet another aspect, the first protein comprises, consists of, or consists essentially of a C-terminal half of a split tobacco etch virus protease and the second protein comprises, consists of, or consists essentially of a complementary N-terminal half of a split tobacco etch virus protease, a protease cleavage site (PCS), and a transcription factor. In some aspects, the N-terminal half of split tobacco etch virus protease comprises, consists of, or consists essentially of SEQ ID NO: 1, 3, 5, or 6. In yet another aspect, the C-terminal half of split tobacco etch virus protease comprises, consists of, or consists essentially of SEQ ID NO: 2, 4, or 7.

In some embodiments, the transcription factor comprises, consists of, or consists essentially of a synthetic transcription (synTF) factor or a naturally occurring transcription factor.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### DESCRIPTION OF THE FIGURES

FIG. 1A shows MESA receptors designed to sense extracellular IL-23. IL-23 nanobodies were grafted onto TMDs from FGFR1 or CD28 and intracellular MESA machinery was used to allow ligand inducible TF liberation and signaling. Most receptor designs resulted in ligand-inducible signaling with fold changes as high a 6 in the presence of IL-23 as shown in FIG. 1B.

FIG. 2 shows 24 possible receptor designs with the experimental design used in FIGS. 1A-1B. Some receptor designs exhibited functional signaling in the presence of IL23, with the best design exhibiting a signal 9.3× higher in the presence of ligand compared to the absence the general flow cytometry gating strategies used. 8 designs were selected for further testing: 1, 9, 11, 12, 16, 17, 20, 24. Samples were run with UltraRainbow Calibration Beads (Spherotech) and fluorescent output normalized across runs to the molecules of equivalent PE-Texas Red (MEPTRs), n=3, *p<0.05 by Student's t-test.

FIG. 3 shows 8 designs selected from FIG. 2 that were selected for further validation. The same cells with a genomically integrated fluorescent reporter were used. Cells were transfected with two receptor chains. A positive control group (secreted ligand) also received the same secreted IL23 plasmid as before. The other groups either received nothing (no ligand) or 250 ng/uL rhIL23 (exogenous ligand) 16 hours after transfection. Media was changed 40 hours after transfection and again supplemented with 250 ng/uL IL23. Cells were harvested for flow cytometry three days after transfection and one design (receptor 12) exhibited a significant increase in signal in the exogenous ligand condition. Samples were run with UltraRainbow Calibration Beads (Spherotech) and fluorescent output normalized across runs to the molecules of equivalent PE-Texas Red (MEPTRs), n=3, *p<0.05 by Student's t-test.

### DETAILED DESCRIPTION

Cell-based therapies represent an exciting frontier in design-driven medicine, leveraging the natural capabilities of cells to sense, process information, and produce and secrete therapeutic molecules in situ. Synthetic receptor systems enable engineered mammalian cell-based therapies to sense physiological cues and produce therapeutic responses. There now exist examples of synthetic receptor systems that can sense surface-bound and soluble extracellular targets and signal through either natural signaling pathways or synthetic gene circuits.

Synthetic sensors have the advantage of minimally disturbing or being regulated by native cellular processes, yet it remains laborious to generate new synthetic receptors for soluble ligands of interest. Although natural receptors exist for many soluble ligands, no systematic strategy has been developed to convert natural receptors into synthetic receptors that signal orthogonally from native pathways.

One such ligand of interest is IL-23, which can be utilized as the target of a synthetic receptor that enables release of a target transcription factor. In utilizing receptor domains and their corresponding biophysical mechanisms, IL-23 can be leveraged and incorporated into a synthetic receptor architecture, particularly by employing the Modular Extracellular Sensor Architecture (MESA), a synthetic receptor system that signals via proteolytic release of a transcription factor upon receptor dimerization. This signaling mechanism enables customized transcriptional output upon detection of the IL-23 and other target ligands. Thus, the present disclosure provides synthetic IL-23 sensors for cell-based therapies, diagnostics, and tools for studying disease pathology.

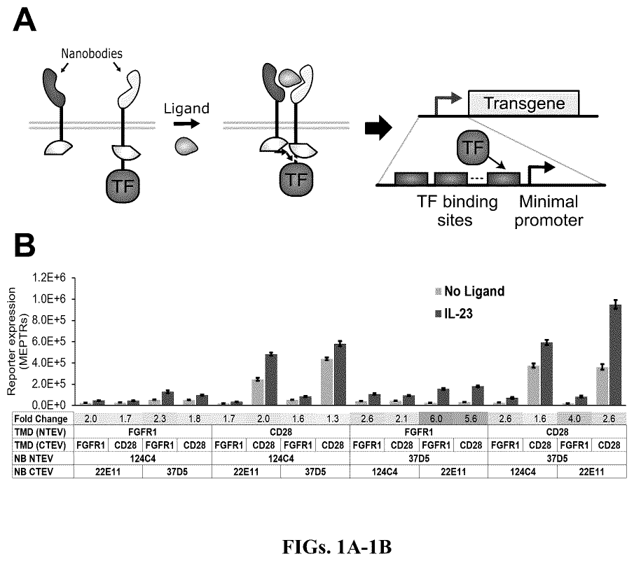

This technology encompasses several synthetic cytokine receptor systems for engineering mammalian cell-based devices to sense soluble, physiological cues. Specifically, this technology employs synthetic receptor domains (i.e.: nanobodies) within the Modular Extracellular Sensor Architecture (MESA) framework to transduce receptor-ligand binding into user defined transcriptional output. MESA receptors comprise transmembrane proteins that are engineered to release a sequestered transcription factor through proteolytic cleavage upon receptor binding to the target ligand (FIG. 1A). Altogether the disclosed technology enables expedient engineering of high surface-expressing synthetic receptors for sensing extracellular, physiological cues.

### I. Definitions

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide.

Regarding proteins, a “deletion” refers to a change in the amino acid sequence that results in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide). A “variant,” “mutant,” or “derivative” of a reference polypeptide sequence may include a deletion relative to the reference polypeptide sequence.

Regarding proteins, “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full-length polypeptide. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

The term “half” is used herein to define a portion of a protein, “split protein”, or nucleic acid sequence that encodes a protein or split protein, wherein the protein or sequence is divided into two parts. The term “half” is non-limiting, in that it does not necessarily defined as being 50% of the split protein. In some instances, half may be any portion, fragment, or percent of the protein. For example, in some embodiments, half of the split protein may comprise 1%, 5%, 10%, 25%, 50%, 75%, 90%, 95% or 99% of the protein. In some embodiments, the split protein may be divided in two halves, such that the first half comprises 1% and the second half comprises 99% of the protein. In some embodiments, the split protein may be divided in two halves, such that the first half comprises 5% and the second half comprises 95% of the protein. In some embodiments, the split protein may be divided in two halves, such that the first half comprises 10% and the second half comprises 90% of the protein. In some embodiments, the split protein may be divided in two halves, such that the first half comprises 25% and the second half comprises 75% of the protein. In some embodiments, the split protein may be divided in two halves, such that the first half comprises 50% and the second half comprises 50% of the protein.

Regarding proteins, the words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

Regarding proteins, the phrases “percent identity” and “% identity,” refer to the percentage of residue matches between at least two amino acid sequences aligned sing a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide.

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acids typically disrupt (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed proteins, mutants, variants, or described herein may have one or more functional or biological activities exhibited by a reference polypeptide (e.g., one or more functional or biological activities exhibited by wild-type protein).

The disclosed proteins may be substantially isolated or purified. The term “substantially isolated or purified” refers to proteins that are removed from their natural environment, and are at least 60% free, preferably at least 75% free, and more preferably at least 90% free, even more preferably at least 95% free from other components with which they are naturally associated.

Also disclosed herein are polynucleotides, for example polynucleotide sequences that encode proteins or polypeptides as disclosed herein. The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

“Transformation” or “transfection” describes a process by which exogenous nucleic acid (e.g., DNA or RNA) is introduced into a recipient cell. Transformation or transfection may occur under natural or artificial conditions according to various methods well known in the art, and may rely on any known method for the insertion of foreign nucleic acid sequences into a prokaryotic or eukaryotic host cell. The method for transformation or transfection is selected based on the type of host cell being transformed and may include, but is not limited to, bacteriophage or viral infection or non-viral delivery. Methods of non-viral delivery of nucleic acids include lipofection, nucleofection, microinjection, electroporation, heat shock, particle bombardment, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term “transformed cells” or “transfected cells” includes stably transformed or transfected cells in which the inserted DNA is capable of replication either as an autonomously replicating plasmid or as part of the host chromosome, as well as transiently transformed or transfected cells which express the inserted DNA or RNA for limited periods of time.

The polynucleotide sequences contemplated herein may be present in expression vectors. For example, the vectors may comprise: (a) a polynucleotide encoding an ORF of a protein; (b) a polynucleotide that expresses an RNA that directs RNA-mediated binding, nicking, and/or cleaving of a target DNA sequence; and both (a) and (b). The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refers to a recombinant nucleic acid that has been engineered to express a heterologous polypeptide (e.g., the fusion proteins disclosed herein). The recombinant nucleic acid typically includes cis-acting elements for expression of the heterologous polypeptide.

Any of the conventional vectors used for expression in eukaryotic cells may be used for directly introducing DNA into a subject. Expression vectors containing regulatory elements from eukaryotic viruses may be used in eukaryotic expression vectors (e.g., vectors containing SV40, CMV, or retroviral promoters or enhancers). Exemplary vectors include those that express proteins under the direction of such promoters as the SV40 early promoter, SV40 later promoter, metallothionein promoter, human cytomegalovirus promoter, murine mammary tumor virus promoter, and Rous sarcoma virus promoter. Expression vectors as contemplated herein may include eukaryotic or prokaryotic control sequences that modulate expression of a heterologous protein (e.g. the fusion protein disclosed herein).

The presently disclosed methods may include delivering one or more polynucleotides, such as or one or more vectors as described herein and/or one or proteins transcribed therefrom, to a host cell. Further contemplated are host cells produced by such methods, and organisms (such as animals, plants, or fungi) comprising or produced from such cells. Conventional viral and non-viral based gene transfer methods can be used to introduce nucleic acids in mammalian cells or target tissues. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell.

In the methods contemplated herein, a host cell may be transiently or non-transiently transfected (i.e., stably transfected) with one or more vectors described herein. In some embodiments, a cell is transfected as it naturally occurs in a subject (i.e., in situ). In some embodiments, a cell that is transfected is taken from a subject (i.e., explanted). In some embodiments, the cell is derived from cells taken from a subject, such as a cell line. Suitable cells may include stem cells (e.g., embryonic stem cells and pluripotent stem cells). A cell transfected with one or more vectors described herein may be used to establish a new cell line comprising one or more vector-derived sequences. In the methods contemplated herein, a cell may be transiently transfected with the components of a system as described herein (such as by transient transfection of one or more vectors, or transfection with RNA), and modified through the activity of a complex, in order to establish a new cell line comprising cells containing the modification but lacking any other exogenous sequence.

As used herein “receptor” refers to synthetic, naturally occurring, or endogenous proteins that are associated with the cell membrane (e.g., membrane bound proteins, integral membrane proteins, transmembrane proteins, glycophosphatidylinosital anchored proteins) and have binding specificity for a cognate ligand, and to proteins having an amino acid sequence which is the same as that of a naturally occurring or endogenous receptor protein (e.g., recombinant proteins, synthetic proteins (i.e., produced using the methods of synthetic organic chemistry)). Accordingly, as defined herein, the term includes mature receptor protein, naturally occurring polymorphic or allelic variants, and other naturally occurring isoforms of a receptor (e.g., produced by alternative splicing or other cellular processes), and modified (e.g. post-translational modifications, lipidated, glycosylated) or unmodified forms of the foregoing. Alternative splicing of RNA encoding a receptor may yield several isoforms of the receptor that differ in the number of amino acids in the protein sequence. These isoforms and other isoforms are expressly encompassed by the term “receptor”. Synthetic, naturally occurring or endogenous receptors can be recovered or isolated from a source which naturally produces the receptor, for example, or produced by synthetic methods.

As used herein, the term “ligand” refers to a compound that comprises at least one peptide, polypeptide, protein moiety that has a binding site with binding specificity for a desired target. For example, the ligand can comprise a (e.g., at least as one) protein moiety (e.g., a dAb) that has a binding site with-binding specificity for a receptor.

The terms “extracellular domain”, “ectodomain” or “ECD” are used interchangeably herein to refer to the extracellular region or a portion thereof exclusive of the transmembrane spanning and cytoplasmic regions. Ligand-binding ectodomains of the receptors described herein sense and bind target ligands including small molecules and proteins. The ectodomains of the receptors described herein may comprise homodimeric and heterodimeric ectodomain binding configurations. Homodimerization and heterodimerization may be ligand-induced.

As used herein, “transmembrane domain” or “TMD” broadly refers to an amino acid sequence of about 15 amino acid residues across the plasma membrane. More preferably, the transmembrane domain comprises at least about 20, 25, 30, 35, 40, or 45 amino acid residues and spans the plasma membrane. The transmembrane domain is abundant within hydrophobic residues and usually has an α-helical structure. In one embodiment, at least 50%, 60%, 70%, 80%, 90%, 95% or more of the amino acids in the transmembrane domain are hydrophobic, such as leucine, isoleucine, tyrosine, or tryptophan.

As used herein, “juxtamembrane domain” or “JMD” refers to an intracellular part of the receptor adjacent to the transmembrane domain.

In some embodiments, the extracellular domain of the human receptor protein is a nanobody that binds to IL-23.

### II. Modular Extracellular Sensor Architecture

The disclosed subject matter relates to integrated “Modular Extracellular Sensor Architecture” (MESA). In some embodiments, a MESA system includes a pair of extracellular receptors where both receptors of the pair contain a ligand binding domain and transmembrane domain, and one receptor contains a protease cleavage site and a functional domain (e.g., transcription regulator such as a transcription regulator that promotes transcription or a transcription regulator that inhibits transcription) and the other receptor contains a protease domain. In some embodiments, a MESA system includes a pair of extracellular receptors where both receptors of the pair contain a ligand binding domain and transmembrane domain, and one receptor contains half of a split protease domain and the other receptor contains the complementary half of the split protease domain as well as a protease cleavage site and a functional domain (e.g., transcription regulator such as a transcription regulator that promotes transcription or a transcription regulator that inhibits transcription). As used herein, a transcription regulator may include a transcription factor that promotes transcription (e.g., by recruiting additional cellular components for transcription) and/or a transcription inhibitor or transcription repressor).

MESA technology and the presently disclosed advancement may be utilized for building living cell-based biosensors. In certain embodiments, MESA technology and the presently disclosed advancement comprise engineered receptor proteins that can detect extracellular ligands (e.g., such as cytokines) and transduce this information across the cell membrane to release an engineered transcription regulator that drives the expression of a user-defined gene.

MESA technology and the presently disclosed advancement have a wide variety of uses including in vitro laboratory assays (e.g., to detect/quantify specific analytes), as powerful new experimental tools for studying in vivo animal models (e.g., wherein engineered cell-based biosensors could be adoptively transferred, generated from transplanted bone marrow, or genetically engineered in a transgenic animal), and as human therapeutics (e.g., for augmenting the functionality of engineered cell-based therapies). MESA technology and the presently disclosed advancement could also be adapted to function in other cell types, such as insect cells or microbes (e.g., yeast) to create cell-based biosensors for a variety of applications.

In certain embodiments of MESA technology and the presently disclosed advancement where two receptors are employed, the general mode of action is that ligand binding induces the aggregation of two or more MESA receptors, bringing an intracellular split protease domain (PR) into proximity with a complementary half of a split protease, leading to reconstitution of the protease and cleavage of the cognate intracellular protease cleavage site (PCS), and upon cleavage of the PCS by PR, a transcription factor (TF) or other functional domain (e.g., a transcription inhibitor) is released from the MESA receptor at the cell membrane to carry out its function (e.g., a TF may localize to the nucleus to induce gene expression). One implementation of this architecture would be a heterodimerization- (or heteromultimerization-) based signaling mechanism. In this system, one engineered receptor chain contains the N-terminal fragment of the split protease and the other engineered receptor chain contains the C-terminal fragment of the split protease as well as the PCS-TF domain. Other implementations include, for example, a system in which one receptor contains a complete protease and the other receptor contains the PCS-TF domain; or a homodimerization- (or homomultimerization-) based mechanism in which each MESA chain contains both PR and PCS-TF domains, but the receptor is engineered such that cleavage may occur in trans, but not in cis (i.e., one chain may not release its own TF).

A general implementation of one embodiment of MESA technology and the presently disclosed advancement is as follows: receptors are designed, DNA sequences encoding these receptors are generated (by molecular biology and/or DNA synthesis) and inserted into a suitable expression vector (such as a plasmid or a stable gene delivery system), the expression vector is transfected or genomically integrated into a suitable cell line or stock of primary cells (together with a suitable reporter construct, which expresses a reporter gene in response to nuclear-localized TF), ligand is added to the cell culture medium, and induced reporter gene expression is quantified by suitable means.

In certain embodiments, MESA technology and the presently disclosed advancement provide a cell-based biosensor for detecting a natural analyte of interest in vitro or in vivo and employing ectodomains (ECDs) to coopt their mechanisms for ligand binding into customized transcriptional outputs.

In some embodiments, MESA technology and the presently disclosed advancement provide: i) an approach where a pair of MESA receptors are engineered with synthetic or naturally occurring ligand-binding domains that recognize a specific peptide; ii) a cell-based biosensor for detecting a specific pattern of multiple analytes of interest (e.g., by coupling MESA receptors to engineered gene circuits to enable signal processing) in vitro; iii) a cell-based biosensor for detecting a specific pattern of multiple analytes of interest (e.g., by coupling MESA receptors to engineered gene circuits to enable signal processing) in vivo; iv) a cell-based biosensor coupled to expression of a gene that enables in vivo imaging (e.g., by MRI) for diagnostic purposes; v) a cell-based biosensor coupled to expression of a therapeutic agent to create targeted cellular therapies, which may be used to treat cancer, autoimmune disease, and other diseases; vi) a multicellular network using synthetic intercellular communication (e.g., engineering some cells to express MESA receptors and others to secrete MESA ligands), with applications including: scientific investigation of biological processes including development, immune function, wound healing, etc., cell & tissue-based products for applications including tissue engineering, regenerative medicine, immune therapy, transplantation medicine, cellular therapies and the like.

All MESA receptors can be modified in order to optimize specific receptor properties. Modifications include, for example, the following: i) varying the length of intracellular spacers (ISP) (on either MESA chain) to include, for example, between 0-20, 0-50, or 0-150 (e.g., 0 . . . 5 . . . 50 . . . 100 . . . 130 . . . or 150) non-structured amino-acid residues (e.g., glycines or alternating glycine-serine residues); ii) varying the predicted mechanical properties of ISP (on either MESA chain) by replacing non-structured amino acids with structured subdomains (e.g., an alpha-helical domain); iii) including an ESP domain (either structured or unstructured, of lengths between 0-20 or 0-50 or 0-150 amino acids). Structured domains may include, for example, an immunoglobulin motif, (e.g., for presentation of ligand-binding (LB) domains that are derived from antibody fragments at a certain distance away from the cell surface); altering the sequence of the PCS to enhance or inhibit the rate of PR-mediated cleavage; or varying the combinations of receptor chains used to constitute a complete MESA receptor system. The transmembrane domain may be derived from either natural or synthetic sequences in order to modulate the kinetics or geometry with which MESA chains associate in the presence or absence of ligand. The specific split TEVp domains on each receptor may also be modified. For example, either of both of the NTEVp and CTEVp domains may comprise mutations from the wildtype TEVp sequence that affect reconstitution propensities, and therefore signaling output from receptors may be modulated. See, Table 1 for wildtype and mutant split TEVp sequences.

MESA variants may use, for example, ligand-binding domain interactions including: i) using an antibody (or a fragment thereof) to bind to the target ligand; ii) implementation in a homodimeric MESA receptor (both antibody fragments are identical and bind to identical sites on a polyvalent ligand, such as a homodimeric cytokine); iii) implementation in a heterodimeric MESA receptor (e.g., each MESA chain incorporates a distinct antibody fragment, such that a monovalent ligand can still induce MESA receptor dimerization or multimerization); iv) incorporating a modular protein-peptide interaction that is not from a receptor ligand system (e.g., conserved protein motifs such as SH3, PDZ, and GBD domains bind distinct and unique consensus peptide motifs) to create an engineered MESA receptor-ligand system.

In certain embodiments, the released functional domain on a MESA receptor (ER-A) is replaced with another functional domain, such as a catalytic domain (whose activity requires cleavage-mediated release), a separate protease domain (whose activity requires cleavage-mediated release), a DNA-binding domain (e.g., zinc-finger or TAL Effector-based domains) coupled to a functional domain (e.g., an endonuclease, a chromatin modifying enzyme such as the Krueppel-associated box or KRAB protein, or other enzymes or cofactor-recruiting domains). In particular embodiments, modification of the MESA system to detect intracellular analytes, such that intracellular versions of the MESA receptors may be: ER-A could contain LB-ISP-PCS-TF domains and its cognate MESA receptor (ER-B) could contain LB-ISP-PR domains; in other embodiments ER-A could contain LB-ISP-PR1-PCS-TF domains and its cognate MESA receptor (ER-B) could contain LB-ISP-PR2 domains, where PR1 and PR2 are portions of a split protease. Ligand-binding by the two chains would again enable protease-mediated cleavage and release of a functional domain (such as transcription factor, TF). In other embodiments, the split protease-based MESA configuration may be modified to detect intracellular ligands.

MESA technology and the presently disclosed advancement may be configured for use in multiple cellular contexts for applications in basic science, biotechnology, and medicine (including both diagnostics and therapeutics). MESA biosensors and the disclosed improvements (e.g., implemented in mammalian cells) would have a wide variety of potential uses including in vitro laboratory assays (e.g., to detect/quantify specific analytes), as powerful new experimental tools for studying in vivo animal models (e.g., engineered cell-based biosensors could be adoptively transferred, generated from transplanted bone marrow, or genetically engineered in a transgenic animal to monitor extracellular species in real time in living animals), and potentially as human therapeutics (e.g., for engineering cell-based therapies that probe their environment and deliver a therapeutic payload only at desirable locations). This powerful synthetic biology technology may also be adapted to function in other cell types, such as insect cells or microbes (e.g., yeast) to create cell-based biosensors for applications in biotechnology.

In MESA technology and the presently disclosed advancement each engineered receptor (ER) of a receptor pair is composed of two chains, each of which is a type I transmembrane protein. The alpha chain (ER-A) may be fused at its C-terminus to one half of a split protease domain, a peptide harboring a TEV protease cleavage site (PCS), and an engineered transcription factor (TF). The beta chain (ER-B) may be fused at its C-terminus to the complementary half of the split TEV protease (PR). Other domains could include ligand-binding domains (LB), extracellular spacers (ESP), intracellular spacers (ISP) (e.g., which may be absent or a short length), transmembrane domains (TM), and juxtamembrane domains (JM). In such embodiments, the binding of ER-A and ER-B to a ligand may lead to receptor oligomerization, PR reconstitution, and PR-mediated cleavage and release of TF. This strategy is suitable for recognition of any ligand possessing more than one domain that may be recognized by a LB domain, as described in detail below. Modular receptor construction is intrinsic MESA technology and the presently disclosed advancement, since receptor design may, in certain embodiments, require adjustment for each receptor-ligand combination. Domain junctions may be engineered by introducing unique restriction sites to facilitate exchange.

MESA technology and the presently disclosed advancement may rely upon the formation of heteromeric complexes. In alternative embodiments, each chain may include both PR and TF domains separated by a PCS and oriented such that each PR domain cleaves in trans but not in cis (i.e., PR cleaves neighboring receptors upon ligand binding-induced aggregation).

In some embodiments, the presently disclosed systems provide cell-based biosensors that perform multifactorial logical evaluation of extracellular signals using the MESA receptors described herein, which transduce extracellular cues into synthetic pathways. Such pathways may be constructed into genetic circuit architectures that can process information in useful ways. For example, one may engineer cells to perform multifactorial evaluations of extracellular inputs using Boolean logic, which is a strategy that has been implemented to date using intracellular sensors. Successful implementation of this strategy using extracellular inputs is an important step toward building mammalian cell-based sensors that interface with natural systems in vivo. For example, initially, three representative types of circuits may be constructed such as “OR”, “NOT IF”, and “AND” gate genetic circuits. Transcriptional control may be implemented using systems known in the art and described herein.

In particular, one may investigate the following circuit architectures, each of which would be useful for probing immune function: a biosensor circuit that reports in response IL-23. After characterizing the qualitative behavior of these circuits, one may also characterize the quantitative function of these circuits. These investigations may be facilitated by choice of engineered transcription factors. Plasmid doses may also be varied to modulate the level of engineered receptor expression. Using these tunable parameters, one may determine the sensitivity of these circuits to various input combinations and strengths (concentrations) and characterize the resulting transfer functions (quantitative relationships between inputs and outputs). In some embodiments, logic gates having multiple inputs may be generated where one input is a ligand as described herein, and the other input is a physiological state (e.g., hypoxia) that effects a response. For example, where the input is the physiological state of hypoxia, a hypoxia-responsive protein/promoter may be used to regulate part of the signaling downstream of MESA, such that the output gene is expressed only under conditions of hypoxia AND in the presence of the ligand for the MESA receptor.

One may also evaluate the dynamic responses of these circuits when extracellular inputs are removed from the system (e.g., by replacing the culture medium). To facilitate these analyses, one may use computational mathematical modeling, as has previously been done for other intracellular genetic circuits. An important extension may be developing systems for stably expressing these circuits and characterizing their performance under these expression conditions. One may need to evaluate the influence of expression on circuit performance, stability, and variability. Strategies for coping with these challenges include expressing both receptor chains (ER-A and ER-B) from a single multicistronic vector, which reduces the number of vectors required. Bicistronic expression would suffice for implementing even the relatively more complicated “AND” gate.

In certain embodiments, MESA technology and the presently disclosed advancement described herein are implemented in mammalian cells, and are employed in any suitable use, such as in vitro laboratory assays (e.g., to detect/quantify specific analytes), as powerful experimental tools for studying in vivo animal models (e.g., engineered cell-based biosensors could be adoptively transferred, generated from transplanted bone marrow, or genetically engineered in a transgenic animal to monitor extracellular species in real time in living animals), and as human therapeutics (e.g., for engineering cell-based therapies that probe their environment and delivery a therapeutic payload only at desirable locations). In other embodiments, MESA technology and the presently disclosed advancement are employed with other cell types, such as insect cells or microbes (e.g., yeast) to create cell-based biosensors for applications in biotechnology.

Any type of suitable ligand binding domain (LB) can be employed with the receptors of MESA technology. Ligand binding domains can, for example, be derived from either an existing receptor ligand-binding domain or from an engineered ligand binding domain. Existing ligand-binding domains could come, for example, from cytokine receptors, chemokine receptors, innate immune receptors (TLRs, etc.), olfactory receptors, steroid and hormone receptors, growth factor receptors, mutant receptors that occur in cancer, neurotransmitter receptors. Engineered ligand-binding domains can be, for example, single-chain antibodies (see scFv constructs discussion below), engineered fibronectin based binding proteins, and engineered consensus-derived binding proteins (e.g., based upon leucine-rich repeats or ankyrin-rich repeats, such as DARPins). The presently disclosed advancement utilizes nanobodies to coopt their mechanisms for ligand binding into customized transcriptional outputs.

Any suitable extracellular spacer (ESP) can be used with the receptors of MESA technology and the presently disclosed advancement. In certain embodiments, the ESP is from 0-30 amino acids long (e.g., 1 . . . 5 . . . 15 . . . 25 . . . or 30), where each amino acid can be, for example, any of the 20 naturally occurring amino acids. In certain embodiments, ESP can be nonstructured or comprised partially or entirely of amino acids predicted to fold into a secondary structure (i.e., an alpha helix) or a tertiary structure. ESP sequences flanking the transmembrane (TM) domain may be selected to adjust the stability of the TM in the membrane (i.e., adding a polar or charged residue to ESP next to (TM) should make it more difficult for that amino acid to be pulled into the membrane). In certain embodiments, ESP is derived from the extracellular portion of a natural receptor sequence.

Any suitable transmembrane domain (TM) can be used with the receptors of MESA technology and the presently disclosed advancement. In certain embodiments, the TM is, for example, a TM domain taken from an existing receptor (e.g., TLR4, CD28, IL-10 receptor, FGFR1, etc.) or engineered using a novel sequence, for example using (TM) consensus sequence features.

Any suitable intracellular spacer (ISP) can be used with the receptors of MESA technology and the presently disclosed advancement. In particular embodiments, no ISP is present. In certain embodiments, the ISP is, for example, 0-30 amino acids long (e.g., 1, 2, 3, 4, 5, 6, . . . 15 . . . 25 . . . or 30 amino acids) where each amino acid can be, for example, any of the 20 naturally occurring amino acids. ISP can be, for example, nonstructured or comprised partially or entirely of amino acids predicted to fold into a secondary structure (i.e., an alpha helix) or a tertiary structure. ISP sequences flanking the TM domain may be selected to adjust the stability of the TM in the membrane (i.e., adding a polar or charged residue to ISP next to (TM) should make it more difficult for that amino acid to be pulled into the membrane). In certain embodiments, ISP is derived from the intracellular portion of a natural receptor sequence.

Any suitable protease cleavage sequence may be employed with the receptors of MESA technology and the presently disclosed advancement. In certain embodiments, the PCSs, for example, are varied by mutating the amino acid at the P1′ position, for example, to any of the 20 amino acids or by introducing 1 or more mutations into the rest of the PCS, e.g., to modify kinetic parameters governing PCS cleavage.

MESA technology and the presently disclosed advancement are not limited to any particular protease or corresponding protease cleavage site. In some embodiments, the protease and cleavage site are from a virus. For example, in certain embodiments, the protease and protease cleavage site are from a virus selected from: tobacco etch virus (TEV), a chymotrypsin-like serine protease and corresponding cleavage sites, alphavirus proteases and cleavage sites, Hepatitis C virus proteases (e.g., N S3 proteases) and corresponding cleavage sites, chymotrypsin-like cysteine proteases and corresponding cleavage sites, papain-like cysteine proteases and cleavage sites, picornavirus leader proteases and cleavage sites, HIV proteases and cleavage sites, Herpesvirus proteases and cleavage sites, and adenovirus proteases and cleavage sites (see, Tong, Chem. Rev. 2002, 102, 4609-4626, herein incorporated by reference in its entirety). In particular embodiments, the proteases and cleavage sites are bacterial in original, such as, for example, from Streptomyces griseus protease A (SGPA), SGPB, and alpha-lytic protease and corresponding cleavage sites. In some embodiments, the proteases and cleavage sites are mammalian. For example, the proteases could be one of the five major classes of proteases known in mammals which include serine proteases, cycteine proteases, metallo proteases, aspartic proteases, and thereonine proteases (see, e.g., Turk et al., The EMBO Journal, 2012, 31, 1630-1643; Lopez-Otin and Overall, 2002, Nat. Rev. Mol. Cell Biol., 2:509-519; Overall and Blobel, 2007, Nat. Rev. Mol. Cell Biol., 8: 245-257; and Lopez-Otin and Bond, 2008, J. Biol. Chem., 283:30422-30437, all of which are herein incorporated in their entireties by references.

In certain embodiments, receptors for MESA technology and the presently disclosed advancement may be designed using engineered ligand binding domains based upon single chain antibody variable fragments (scFv). The loop linking heavy and light chain-derived fragments of an scFv may be designed (both in length and sequence) to favor monomeric scFvs, dimeric scFvs, trimeric scFvs, etc. Loop length may be, for example, 0-30 amino acids long, where each amino acid may be, for example, any of the 20 naturally occurring amino acids. One may select a loop to favor scFvs or to favor homomultimeric scFvs. ScFv may be engineered, for example, from isolated antibody, BCR, or TCR sequences, or they may be isolated from a random library, such as phage-display, bacterial-display, or yeast-display. In other embodiments, receptors for MESA technology and the presently disclosed advancement may be designed using engineered ligand binding domains based upon a camelid antibody analog termed a “nanobody.”

In certain embodiments, directed evolution could be used to optimize performance characteristics of receptors including, for example: low background signaling, enhanced signal-to-noise ratio, enhanced sensitivity for low ligand concentrations, and enhanced dynamic range (differential responsiveness over a wider range of ligand concentrations). Directed evolution could be performed, for example, by a scheme in which (a) a library of genetic variants upon an initial receptor design are created (b) each variant is expressed in a separate cell (c) this pool of cells is exposed to a functional screen to either eliminate cells (and therefore receptor variants) exhibiting undesirable activity or retain cells (and therefore receptor variants) that exhibit some desirable activity. This process could be repeated to enrich for variants with desirable properties. A variation upon this method would be to isolate variants in this fashion after 1 or more rounds of enrichment, introduce additional genetic diversity into this library, and return to the cell-based screening; this could be repeated for multiple rounds until the pool or individual constructs within the pool exhibit properties that meet some threshold for considering it a success. In some embodiments, one could (a) generate a library of DNA sequences encoding MESA variants using error-prone PCR or other molecular biology techniques to incorporate chemically synthesized DNA oligonucleotides including variation at defined positions; variation could be introduced at ISP, PCS, PR, TM, ESP, LB, or combinations of these sites, (b) each variant could be cloned into an expression vector based upon adeno-associated virus (AAV), viral vectors could be packaged by standard techniques, and AAV vectors could be used to transduce cells at a ratio of viruses to cells such that each cell expresses only one variant of the MESA library, and then (c) this pool of cells that expresses the MESA library (one variant per cell) could be used for cell-based assays; for example, cells could be transfected or transduced with a reporter construct that reads out MESA signaling by inducing expression of a fluorescent protein, and then the MESA pool of reporter-bearing cells could be sorted using fluorescence assisted cell sorting (FACS) based upon whether the reporter construct is induced or not when exposed to zero ligand or some finite quantity of ligand.

### III. MESA System (MESA)

To build receptors that sense soluble, physiological cues and produce user-defined transcriptional output in response, the Modular Extracellular Sensor Architecture (MESA) was used (Table 1) (Daringer, et al. ACS Synthetic Biology, 2014; Dolberg, et al. Nature Chemical Biology, 2021). MESA receptors comprise two types of transmembrane proteins that are engineered to associate upon ligand binding and release a sequestered transcription factor. Both MESA receptor types typically contain a signal peptide, an optional 3×FLAG epitope tag, extracellular domains that bind to the target ligand, and a transmembrane domain. Inside the cell membrane, one type contains the C-terminal half of a split tobacco etch virus (TEV) protease, a protease recognition sequence, and an intracellular transcription factor. The second type contains the N-terminal half of the split TEV protease inside the cell. Upon extracellular ligand binding, the two receptor types are driven to reconstitute the intracellular split protease and release the tethered transcription factor to translocate to the nucleus and activate target gene expression. Published versions of MESA receptors contain small molecule binding domains, scFvs, and nanobodies as extracellular ligand binding domains (Edelstein, et al. Synthetic Biology, 2020; Dolberg, et al. Nature Chemical Biology, 2021). In embodiments described herein, natural receptor ectodomains were identified, which may also be used to confer MESA receptor binding to their cognate ligands, with the specific construction dependent on the biophysical ligand-binding mechanism of the natural parental receptor system. Wildtype and mutant MESA sequences can be found in Table 1.

TABLE 1
Sequences of the intracellular binding domain

| Name | SEQ ID NO | Sequence |
|---|---|---|
| NTEVp wildtype | 1 | ESLFKGPRDYNPISSTICHLTNESDGHTTSLYGIG |
| | | FGPFIITNKHLFRRNNGTLLVQSLHGVFKVKNT |
| | | TTLQQHLIDGRDMIIIRMPKDFPPFPQKLKFREP |
| | | QREERICLVTTNFQT |
| CTEVp wildtype | 2 | KSMSSMVSDTSCTFPSSDGIFWKHWIQTKDGQ |
| | | CGSPLVSTRDGFIVGIHSASNFTNTNNYFTSVPK |
| | | NFMELLTNQEAQQWVSGWRLNADSVLWGGH |
| | | KVFMVKPEEPFQPVKEATQLMN |
| NTEVp 75S | 3 | ESLFKGPRDYNPISSTICHLTNESDGHTTSLYGIG |
| | | FGPFIITNKHLFRRNNGTLLVQSLHGVFKVKNT |
| | | TTLQQSLIDGRDMIIIRMPKDFPPFPQKLKFREP |
| | | QREERICLVTTNFQT |
| CTEVp 190K | 4 | KSMSSMVSDTSCTFPSSDGIFWKHWIQTKDGQ |
| | | CGSPLVSTRDGFIVGIHSASNFTNTNNYFTSVPK |
| | | NFMELKTNQEAQQWVSGWRLNADSVLWGGH |
| | | KVFMVKPEEPFQPVKEATQLMN |
| NTEVp 103H | 5 | ESLFKGPRDYNPISSTICHLTNESDGHTTSLYGIG |
| | | FGPFIITNKHLFRRNNGTLLVQSLHGVFKVKNT |
| | | TTLQQHLIDGRDMIIIRMPKDFPPFPQKLKFREH |
| | | QREERICLVTTNFQT |
| NTEVp 75E | 6 | ESLFKGPRDYNPISSTICHLTNESDGHTTSLYGIG |
| | | FGPFIITNKHLFRRNNGTLLVQSLHGVFKVKNT |
| | | TTLQQELIDGRDMIIIRMPKDFPPFPQKLKFREP |
| | | QREERICLVTTNFQT |
| CTEVp 158P | 7 | KSMSSMVSDTSCTFPSSDGIFWKHWIQTKDGQ |
| | | CGSPLVSPRDGFIVGIHSASNFTNTNNYFTSVPK |
| | | NFMELLTNQEAQQWVSGWRLNADSVLWGGH |
| | | KVFMVKPEEPFQPVKEATQLMN |
| Protease | 8 | ENLYFQM |
| cleavage site | | |
| (PCS) | | |
| Functional | 9 | PKKKRKVSGDALDDFDLDMLGSDALDDFDLD |
| domain | | MLGSDALDDFDLDMLGSDALDDFDLDMLGSG |
| | | GGGSGGGGSGGGGSGTARPGERPFQCRICMRN |
| | | FSKGERLVRHTRTHTGEKPFQCRICMRNFSRMD |
| | | NLSTHLRTHTGEKPFQCRICMRNFSRKDALNRH |
| | | LKTHLRGS |

The receptor systems chosen included hetero-associative (different receptor chains associate) and homo-associative (like receptor chains associate) ligand-dependent signaling mechanisms between two or more receptor chains, making them promising candidates for use in a ligand-mediated split protease reconstitution and trans-cleavage (MESA-like) signaling mechanism.

The disclosed sensors and systems can be utilized for, among other thing, cell-based therapies in which the sensor detects a disease marker of interest and response by regulating production of a therapeutic output; cell-based; diagnostics/theragnostics to report on presence or relative abundance of a disease marker of interest by regulating production of a reporter gene; and products enabling fundamental research on the dynamics of selected disease markers through the course of disease or in in vitro laboratory assays to detect and quantify target ligands.

Unlike the natural receptors from which these synthetic sensors are derived, the disclosed sensors signal through orthogonal signaling pathways that are self-contained and have the advantage of minimally disturbing or being regulated by native cellular processes. In particular, these sensors can signal via transcription factors of the Composable Mammalian Elements of Transcription (COMET), a panel of synthetic zinc finger-based transcription factors, which makes them easier to use with sophisticated downstream circuitry. These receptors are also amenable to other user-defined synTFs besides those from the COMET toolkit.

The disclosed sensors demonstrate substantially higher surface expression, leading to improved ability to sense ligands outside of the cell compared to previously-reported sensors. The strategy employed here identifies the most important aspects of design of synthetic receptors for sensing IL-23 to propose generalizable rules for this process.

In sum, the present disclosure provides cell-based biosensing capabilities to engineer cell-based devices to sense and respond to physiological ligand targets. In particular, the present disclosure includes engineering custom transcriptional output upon detection of tumor microenvironment cues, detection of general inflammation, and detection of general immune system activation (i.e.: IL-23). Accordingly, this technology has value for many types of cell-based therapies, diagnostics, and research tools.

### IV. Interleukin-23 (IL-23)

Interleukin-23 (IL-23) is an inflammatory cytokine, and its dysregulation is implicated in many diseases. IL-23 is secreted by activated immune cells and includes an IL12B subunit and an IL-23A subunit. IL-23 is a heterodimer that binds to the receptor complex including IL-23R and IL-12RB1. Receptor binding leads to the recruitment of Janus kinase 2 as well as tyrosine kinase 2 kinases. IL-23 imbalance and increase is associated with autoimmune and inflammatory diseases as well as cancer. IL-23 is known to facilitate the development of inflammation in numerous models, including arthritis, intestinal inflammation, and psoriasis. Further low concentrations of IL-23 support lung tumor growth, whereas high concentrations inhibit proliferation of lung cancer cells. Because of presence of multiple binding epitopes and the mechanistic features of IL-23, it was hypothesized that IL-23 could induce association of MESA receptors and resulting split TEVp reconstitution and cleavage.

### V. Extracellular Domains

The biosensors may incorporate one or more extracellular domains. In some aspects, the biosensors include an extracellular domain comprising, consisting of, or consisting essentially of a nanobody comprising, consisting of, or consisting essentially of a binding domain that specifically binds to IL-23.

Exemplary extracellular domains can be found in Table 2 below:

TABLE 2
Sequences of the Extracellular Domain Nanobodies

| | SEQ | |
|---|---|---|
| Name | ID NO | Sequence |
| 124c4 | 10 | EVQLVESGGGLVQPGGSLRLSCAASGFTLDDY |
| | | AIAWFRQAPGKEREGVSGIDSGDGSAYYADSV |
| | | KGRFTISSDNAKNTVYLQMNSLRPEDTAVYYC |
| | | ARVRTGWGLNAPDYAMDYWGKGTLVTVSS |
| 22e11 | 11 | EVQLVESGGGLVQAGGSLRLSCAASGRTFSWS |
| | | AVGWFRQAPGKEREFVAAIRWSGGSPYYADSV |
| | | KDRFTISRDNAKNTVYLQMNSLRPEDTAVYLC |
| | | GETSLFPTSRGSHYDTWGQGTQVTVSS |
| 37d5 | 12 | EVQLVESGGGLVQPGGSLRLSCAASGFTLDYL |
| | | AIGWFRQAPGKEREGVSCVSSSGQYTYYADSV |
| | | KGRFTISRDNAESTVYLQMNSLKPEDTAVYYC |
| | | ATDPECYRVRGYYNGEYDYWGQGTQVTVSS |

In some aspects, the biosensors include a first and a second protein each comprising an extracellular domain. The extracellular domain of the first protein may comprise a first nanobody that specifically binds to IL-23. The extracellular domain of the second protein comprises a second nanobody that specifically binds to IL-23. In some aspects, the nanobodies comprise, consist of, or consist essentially of an amino acid sequence SEQ ID NOs: 10-12. In some aspects, the first nanobody comprises a different amino acid sequence than the second nanobody. In some aspects, the first nanobody comprises the same amino acid sequence of the second nanobody. In some aspects, the nanobodies bind to separate ligands of IL-23.

### VI. EXAMPLES

### Re-Engineering a Minimal MESA for Detecting IL-23

Applicant chose to modify an existing MESA platform to sense IL-23. Each MESA receptor comprises intracellular components described above, a transmembrane domain (either derived from CD28 or FGFR1), and an extracellular domain consisting of a nanobody specific for IL-23 (FIG. 1A). Ultimately, Applicant chose three published nanobody sequences to include, based on the fact that they demonstrated high affinity and each bound unique epitopes. This architecture results an array of possible receptor chains (one ECD, one TMD, and either N-terminal split TEV protease (N-TEVp) or C-terminal split TEV protease (C-TEVp)) of approximately 50 kDa which can be paired up with the complementary chain that binds a different epitope. Applicant tested these designs in high-throughput, by leveraging cells with a genomically integrated fluorescent reporter under a COMET promoter. Many of the engineered IL-23 receptors functioned successfully, with a maximal fold induction of approximately 6 in the presence of ligand. For brevity, a subset of inducible designs is shown (FIG. 1).

As, shown in FIGS. 1A-1B. Synthetic receptors were designed on the modular extracellular sensing architecture (MESA) platform to detect interleukin 23 (IL-23). Each receptor comprises two chains that dimerize upon ligand binding to enable downstream signaling (A). Receptor chains include an ectodomain consisting of a nanobody specific for IL-23 (124c4, 22e11, or 37D5), a transmembrane domain (either derived from CD28 or FGFR1), and half of the necessary intracellular machinery from the MESA platform. The intracellular machinery includes a split protease derived from tobacco etch virus (TEVp). The chain containing the C-terminal portion of the protease (C-TEVp) also contains an intracellular COMET transcription factor (TF) that is initially sequestered at the plasma membrane. Upon ligand binding and chain dimerization, the TEVp reconstitutes, cleaves a target sequence, and liberates the TF for signaling. This architecture results an array of possible receptor chains (one ECD, one TMD, and either N-terminal split TEV protease (N-TEVp) or C-terminal split TEV protease (C-TEVp)) which can be paired up with the complementary chain that binds a different epitope. We tested a subset of these designs in high-throughput, by leveraging HEK cells with a genomically integrated fluorescent reporter under a COMET promoter. Cells were transfected with two receptor chains (displayed in the table below the graph), and in the IL-23 conditions were also transfected with a plasmid encoding for a secreted hIL23. After two days, cells were harvested and analyzed by flow cytometry. Many of the engineered IL-23 receptors functioned successfully, with a maximal fold induction of approximately 6 in the presence of ligand. Samples were run with UltraRainbow Calibration Beads (Spherotech) and fluorescent output normalized across runs to the molecules of equivalent PE-Texas Red (MEPTRs), n=3.

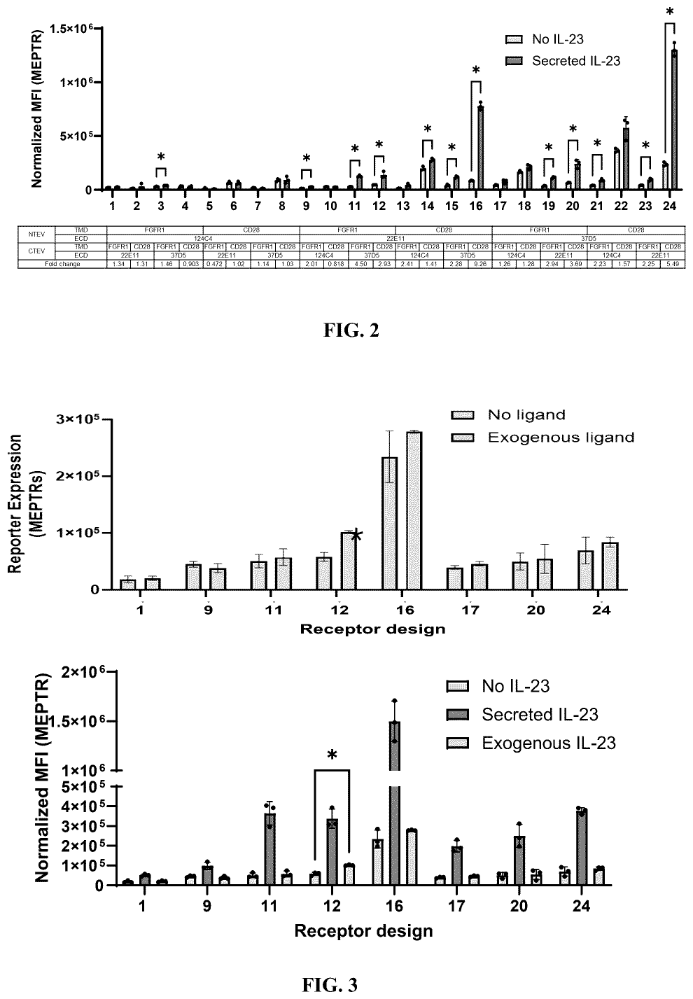

In subsequent experiments, each receptor design (24 total possible combinations) was implemented, with the same experimental design used in FIG. 1. Many designs exhibited functional signaling in the presence of IL23, with the best design exhibiting a signal 9.3× higher in the presence of ligand compared to the absence. Different receptor designs yielded varying fold induction, background intensity, and maximal signal induction (FIG. 2). Ultimately 8 designs were selected for further testing: 1, 9, 11, 12, 16, 17, 20, 24. Samples were run with UltraRainbow Calibration Beads (Spherotech) and fluorescent output normalized across runs to the molecules of equivalent PE-Texas Red (MEPTRs), n=3, *p<0.05 by Applicant's t-test.

Eight designs from FIG. 2 were selected for further validation. In this experiment, the same cells with a genomically integrated fluorescent reporter were used. Cells were transfected with two receptor chains. A positive control group (secreted ligand) also received the same secreted IL23 plasmid as before. The other groups either received nothing (no ligand) or 250 ng/uL rhIL23 (exogenous ligand) 16 hours after transfection. Media was changed 40 hours after transfection and again supplemented with 250 ng/uL IL23. Cells were harvested for flow cytometry three days after transfection and one design (receptor 12) exhibited a significant increase in signal in the exogenous ligand condition (FIG. 3). Samples were run with UltraRainbow Calibration Beads (Spherotech) and fluorescent output normalized across runs to the molecules of equivalent PE-Texas Red (MEPTRs), n=3, *p<0.05 by Applicant's t-test.

### Example 2—Design and Prototype Receptors

Plasmids are assembled for individual receptor chains by swapping out genes for TMDs and ECDs in existing MESA receptor designs. Using digest-ligate molecular cloning, plasmids are assembled such that each includes genes for either N-TEV or C-TEV MESA components described above with varying combinations of ECDs and TMDs. FGFR1 and CD28 transmembrane domains are tested as they exhibited the greatest signal induction in the presence of ligand (compared to no ligand) and highest SNR when paired together in previous work. Nanobodies are tested as the extracellular binding domains with published sequences (124C4, 22E11, 37D5, sequences provided in Table 2). Nanobodies are antibody fragments produced by camelids derived from heavy-chain only IgG antibodies. Nanobodies have been explored for use as drug therapies and in electric biosensors due to their smaller size and chemical stability. Recent improvements in in vitro nanobody production through directed evolution make them an appealing choice of ECD in synthetic receptor development. The nanobodies have each been studied as possible inhibitors of IL-23 by binding to either the p19 or the p40 domains. Each ECD has an attached Myc protein tag for later antibody staining. Genes for a corresponding fluorescent reporter are situated under the COMET promoter that responds bio-orthogonally to the TF. All new gene constructs are verified with whole plasmid sequencing. HEK293FT cell lines with genomically integrated DsRedExpress2 fluorescent reporter are provided, which is transcribed in response to the COMET TF.

### Example 3—Evaluation of Basal Receptor Signaling Competency

Combinatorial receptor designs undergo high-throughput screenings for 1) surface expression, 2) activation with secreted ligand, and 3) activation with exogenous ligand. Evaluating receptor function at each of these stages sequentially and eliminating candidates that do not pass screening criteria allows Applicant to conserve time and resources while still assessing a large pool of candidates. The surface expression of a cell surface receptor is fundamental to its purpose. Similarly, if a receptor does not function in conditions of high ligand concentration (secreted ligand), it is unlikely to function in conditions of lower ligand concentration (exogenous ligand). To measure surface expression, each design is transiently cotransfected into HEK293FT cells using polyethylenimine (PEI). PEI is a cationic polymer that forms a stable complex with DNA and is a highly efficient and viral-free vector for plasmid transfection. Plasmids for NTEV and CTEV chain pairs are complexed together with PEI before being administered to HEK293FT cells. Cells are harvested and stained with a fluorescent anti-Myc antibody, and analyzed with flow cytometry. For functional tests, plasmids are transiently co-transfected for paired receptor chains with genes for secreted human IL-23, or incubated with recombinant human IL-23-supplemented media, into a HEK293FT cell line with the genomically-integrated DsRedExpress2 reporter. Applicant harvests transfected cells 2-3 days after gene delivery, and measure receptor activation via flow cytometry.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

All patents, patent applications, provisional applications, and publications referred to or cited herein are incorporated by reference in their entirety, including all figures and tables, to the extent they are not inconsistent with the explicit teachings of this specification.

What is claimed is:

1. An interleukin 23 (IL-23) receptor, comprising a protein dimer including a first protein and a second protein each comprising:
(a) an IL-23 extracellular ligand-binding domain,
(b) a transmembrane domain,
(c) an intracellular dimerizing domain;
wherein the intracellular dimerizing domain of the first protein comprises a first half of a split protease; and
wherein the intracellular dimerizing domain of the second protein comprises (i) a complementary second half of the split protease, (ii) a protease cleavage site (PCS), and (iii) a transcription factor linked thereto.

2. The IL-23 receptor of claim 1, wherein the split protease components reconstitute upon dimerization of the first protein and the second protein, cleaving the PCS and releasing the transcription factor.

3. The IL-23 receptor of claim 1, wherein the first protein and second protein each further comprise a juxtamembrane domain comprising 5-12 amino acids connected to a cytoplasmic end of the transmembrane domain.

4. The IL-23 receptor of claim 1, wherein the first protein, the second protein, or both further comprise a signal peptide, which is, optionally, derived from a human CD8a receptor or a human IgG variable heavy chain.

5. The IL-23 receptor of claim 1, wherein the extracellular domain of the first protein comprises a first nanobody comprising a binding domain that specifically binds to IL-23, and the extracellular domain of the second protein comprises a second nanobody comprising a binding domain that specifically binds to 11-23.

6. The IL-23 receptor of claim 5, wherein the first nanobody binds to a first IL-23 epitope and the second nanobody binds to the first IL-23 or a second IL-23 epitope.

7. The IL-23 receptor of claim 5, wherein the first nanobody comprises an amino acid sequence selected from SEQ ID NO: 10-12, and wherein the second nanobody comprises an amino acid sequence selected from SEQ ID NOs: 10-12, and optionally, wherein the second nanobody comprises a different amino acid sequence than the first nanobody.

8. The IL-23 receptor of claim 3, wherein the extracellular domain, the transmembrane domain, and the juxtamembrane domain are all derived from the same human protein.

9. The IL-23 receptor of claim 3, wherein the extracellular domain, the transmembrane domain, and the juxtamembrane domain are derived from at least two different human proteins.

10. The IL-23 receptor of claim 1, wherein the transmembrane domain is derived from a murine or human CD28 receptor or a FGFR1 receptor.

11. The IL-23 receptor of claim 1, wherein the juxtamembrane domain comprises a flexible repeated sequence of glycine and serine amino acids.

12. The IL-23 receptor of claim 1, wherein the first protein comprises an N-terminal half of a split tobacco etch virus protease and the second protein comprises a complementary C-terminal half of a split tobacco etch virus protease, a protease cleavage site (PCS), and a transcription factor.

13. The IL-23 receptor of claim 1, wherein the first protein comprises a C-terminal half of a split tobacco etch virus protease and the second protein comprises a complementary N-terminal half of a split tobacco etch virus protease, a protease cleavage site (PCS), and a transcription factor.

14. The IL-23 receptor of claim 12, wherein the N-terminal half of split tobacco etch virus protease comprises SEQ ID NO: 1, 3, 5, or 6.

15. The IL-23 receptor of claim 12, wherein the C-terminal half of split tobacco etch virus protease comprises SEQ ID NO: 2, 4, or 7.

16. The IL-23 receptor of claim 1, wherein the transcription factor is a synthetic transcription (synTF) factor or a naturally occurring transcription factor.


SEQUENCE LISTING (12 sequences)

SEQ ID NO: 1 - protein, 117 aa, unidentified
ESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQT

SEQ ID NO: 2 - protein, 118 aa, unidentified
KSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMN

SEQ ID NO: 3 - protein, 117 aa, synthetic construct
ESLFKGPRDYNPISSTICHLTNESDGHTTSLYGIGFGPFIITNKHLFRRNNGTLLVQSLHGVFKVKNTTTLQQSLIDGRDMIIIRMPKDFPPFPQKLKFREPQREERICLVTTNFQT

SEQ ID NO: 4 - protein, 118 aa, synthetic construct
KSMSSMVSDTSCTFPSSDGIFWKHWIQTKDGQCGSPLVSTRDGFIVGIHSASNFTNTNNYFTSVPKNFMELKTNQEAQQWVSGWRLNADSVLWGGHKVFMVKPEEPFQPVKEATQLMN

SEQ ID NO: 5 - protein, 117 aa, synthetic construct
ESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREHQREERICLVTTNFQT

SEQ ID NO: 6 - protein, 117 aa, synthetic construct
ESLFKGPRDYNPISSTICHLTNESDGHTTSLYGIGFGPFIITNKHLFRRNNGTLLVQSLHGVFKVKNTTTLQQELIDGRDMIIIRMPKDFPPFPQKLKFREPQREERICLVTTNFQT

SEQ ID NO: 7 - protein, 118 aa, synthetic construct
KSMSSMVSDTSCTFPSSDGIFWKHWIQTKDGQCGSPLVSPRDGFIVGIHSASNFTNTNNYFTSVPKNFMELLTNQEAQQWVSGWRLNADSVLWGGHKVFMVKPEEPFQPVKEATQLMN

SEQ ID NO: 8 - protein, 7 aa, synthetic construct
ENLYFQM

SEQ ID NO: 9 - protein, 168 aa, synthetic construct
PKKKRKVSGDALDDFDLDMLGSDALDDFDLDMLGSDALDDFDLDMLGSDALDDFDLDMLGSGGGGSGGGGSGGGGSGTARPGERPFQCRICMRNFSKGERLVRHTRTHTGEKPFQCRICMRNFSRMDNLSTHLRTHTGEKPFQCRICMRNFSRKDALNRHLKTHLRGS

SEQ ID NO: 10 - protein, 125 aa, synthetic construct
EVQLVESGGGLVQPGGSLRLSCAASGFTLDDYAIAWFRQAPGKEREGVSGIDSGDGSAYYADSVKGRFTISSDNAKNTVYLQMNSLRPEDTAVYYCARVRTGWGLNAPDYAMDYWGKGTLVTVSS

SEQ ID NO: 11 - protein, 123 aa, synthetic construct
EVQLVESGGGLVQAGGSLRLSCAASGRTFSWSAVGWFRQAPGKEREFVAAIRWSGGSPYYADSVKDRFTISRDNAKNTVYLQMNSLRPEDTAVYLCGETSLFPTSRGSHYDTWGQGTQVTVSS

SEQ ID NO: 12 - protein, 126 aa, synthetic construct
EVQLVESGGGLVQPGGSLRLSCAASGFTLDYLAIGWFRQAPGKEREGVSCVSSSGQYTYYADSVKGRFTISRDNAESTVYLQMNSLKPEDTAVYYCATDPECYRVRGYYNGEYDYWGQGTQVTVSS